(19) United States
(12) Patent Application Publication
(10) Pub. No.: US 2026/0020899 A1
(43) Pub. Date: Jan. 22, 2026

(54) SURGICAL INSTRUMENTS HAVING NON-LINEAR CAM SLOTS

(71) Applicant: INTUITIVE SURGICAL OPERATIONS, INC., Sunnyvale, CA (US)

(72) Inventor: Iman Khalaji, San Jose, CA (US)

(73) Assignee: INTUITIVE SURGICAL OPERATIONS, INC., Sunnyvale, CA (US)

(21) Appl. No.: 19/341,392

(22) Filed: Sep. 26, 2025

Related U.S. Application Data
(63) Continuation of application No. 17/081,088, filed on Oct. 27, 2020.
(60) Provisional application No. 62/947,307, filed on Dec. 12, 2019.
(60) Provisional application No. 62/947,263, filed on Dec. 12, 2019.

(51) Int. Cl. A61B 18/14; A61B 17/00; A61B 17/29; A61B 17/295; A61B 18/00; A61B 18/12; A61B 34/00; A61B 34/30; A61B 34/35

(52) U.S. Cl. CPC A61B 18/1447; A61B 18/1445; A61B 34/30; A61B 2017/00314; A61B 2017/00353; A61B 17/2909; A61B 2017/2932; A61B 2017/2933; A61B 2017/2934; A61B 2017/2936; A61B 2017/2944; A61B 17/295; A61B 2018/00077; A61B 2018/00083; A61B 2018/00589; A61B 2018/00601; A61B 2018/0063; A61B 2018/126; A61B 2018/1452; A61B 2018/1467; A61B 18/1482; A61B 2034/306; A61B 34/35; A61B 34/71

(57) ABSTRACT

A surgical instrument includes an end effector with opposing jaws that open and close relative to each other. The jaws each include a cam slot for receiving a cam pin. Translation of the pin through the cam slots opens and closes the jaws. The cam slots are shaped such that at least one of the jaws applies a grip force that is substantially proportional to a force applied to the pin to translate the pin through at least a portion of the slots (i.e., the ratio between force input and the resulting force output remains substantially the same as the pin travels through at least one portion of the slots). This design provides a constant mechanical advantage between the force applied to the pin and the force applied by the jaws, thereby allowing a user to more easily regulate the forces applied to tissue held by the jaws.

### CROSS-REFERENCE TO RELATED APPLICATIONS

This application is a continuation of U.S. application Ser. No. 17/081,088 filed Oct. 27, 2020 which claims the benefit of U.S. Provisional Application Ser. No. 62/947,307, filed Dec. 12, 2019 and U.S. Provisional Application Ser. No. 62/947,263, filed Dec. 12, 2019, the entire disclosures of which are incorporated herein by reference for all purposes.

### BACKGROUND

The field of the present disclosure relates to medical instruments, and more particularly to surgical instruments with opposing jaws that apply sufficient gripping forces to handle, seal, staple or cut tissue and vessels of varying size and diameter.

Minimally invasive medical techniques are intended to reduce the amount of extraneous tissue that is damaged during diagnostic or surgical procedures, thereby reducing patient recovery time, discomfort, and deleterious side effects. One effect of minimally invasive surgery, for example, is reduced post-operative hospital recovery times. The average hospital stay for a standard open surgery is typically significantly longer than the average stay for an analogous minimally invasive surgery (MIS). Thus, increased use of MIS could save millions of dollars in hospital costs each year. While many of the surgeries performed each year in the United States could potentially be performed in a minimally invasive manner, only a portion of the current surgeries uses these advantageous techniques due to limitations in minimally invasive surgical instruments and the additional surgical training involved in mastering them.

Improved surgical instruments such as tissue access, navigation, dissection and sealing instruments have enabled MIS to redefine the field of surgery. These instruments allow surgeries and diagnostic procedures to be performed with reduced trauma to the patient. A common form of minimally invasive surgery is endoscopy, and a common form of endoscopy is laparoscopy, which is minimally invasive inspection and surgery inside the abdominal cavity. In standard laparoscopic surgery, a patient's abdomen is insufflated with gas, and cannula sleeves are passed through small (approximately one-half inch or less) incisions to provide entry ports for laparoscopic instruments.

Laparoscopic surgical instruments generally include an endoscope (e.g., laparoscope) for viewing the surgical field and tools for working at the surgical site. The working tools are typically similar to those used in conventional (open) surgery, except that the working end or end effector of each tool is separated from its handle by an extension tube (also known as, e.g., an instrument shaft or a main shaft). The end effector can include, for example, a clamp, grasper, scissor, stapler, cautery tool, linear cutter, or needle holder.

To perform surgical procedures, the surgeon passes working tools through cannula sleeves to an internal surgical site and manipulates them from outside the abdomen. The surgeon views the procedure from a monitor that displays an image of the surgical site taken from the endoscope. Similar endoscopic techniques are employed in, for example, arthroscopy, retroperitoneoscopy, pelviscopy, nephroscopy, cystoscopy, cisternoscopy, sinoscopy, hysteroscopy, urethroscopy, and the like.

Minimally invasive telesurgical robotic systems are being developed to increase a surgeon's dexterity when working on an internal surgical site, as well as to allow a surgeon to operate on a patient from a remote location (outside the sterile field). In a telesurgery system, the surgeon is often provided with an image of the surgical site at a control console. While viewing a three dimensional image of the surgical site on a suitable viewer or display, the surgeon performs the surgical procedures on the patient by manipulating master input or control devices of the control console, which in turn control motion of the servo-mechanically operated slave instruments.

The servomechanism used for telesurgery will often accept input from two master controllers (one for each of the surgeon's hands) and may include two or more robotic arms on each of which a surgical instrument is mounted. Operative communication between master controllers and associated robotic arm and instrument assemblies is typically achieved through a control system. The control system typically includes at least one processor that relays input commands from the master controllers to the associated robotic arm and instrument assemblies and back from the instrument and arm assemblies to the associated master controllers in the case of, for example, force feedback or the like. One example of a robotic surgical system is the DA VINCI™ system commercialized by Intuitive Surgical, Inc. of Sunnyvale, Calif.

A variety of structural arrangements have been used to support the surgical instrument at the surgical site during robotic surgery. The driven linkage or “slave” is often called a robotic surgical manipulator, and exemplary linkage arrangements for use as a robotic surgical manipulator during minimally invasive robotic surgery are described in U.S. Pat. Nos. 7,594,912 (filed Sep. 30, 2004), 6,758,843 (filed Apr. 26, 2002), 6,246,200 (filed Aug. 3, 1999), and 5,800,423 (filed Jul. 20, 1995), the full disclosures of which are incorporated herein by reference in their entirety for all purposes. These linkages often manipulate an instrument holder to which an instrument having a shaft is mounted. Such a manipulator structure can include a parallelogram linkage portion that generates motion of the instrument holder that is limited to rotation about a pitch axis that intersects a remote center of manipulation located along the length of the instrument shaft. Such a manipulator structure can also include a yaw joint that generates motion of the instrument holder that is limited to rotation about a yaw axis that is perpendicular to the pitch axis and that also intersects the remote center of manipulation. By aligning the remote center of manipulation with the incision point to the internal surgical site (for example, with a trocar or cannula at an abdominal wall during laparoscopic surgery), an end effector of the surgical instrument can be positioned safely by moving the proximal end of the shaft using the manipulator linkage without imposing potentially hazardous forces against the abdominal wall. Alternative manipulator structures are described, for example, in U.S. Pat. Nos. 6,702,805 (filed Nov. 9, 2000), 6,676,669 (filed Jan. 16, 2002), 5,855,583 (filed Nov. 22, 1996), 5,808,665 (filed Sep. 9, 1996), 5,445,166 (filed Apr. 6, 1994), and 5,184,601 (filed Aug. 5, 1991), the full disclosures of which are incorporated herein by reference in their entirety for all purposes.

During the surgical procedure, the telesurgical system can provide mechanical actuation and control of a variety of surgical instruments or tools having end effectors that perform various functions for the surgeon, for example, holding or driving a needle, grasping a blood vessel, dissecting tissue, or the like, in response to manipulation of the master input devices. Manipulation and control of these end effectors is a particularly beneficial aspect of robotic surgical systems. Such mechanisms should be appropriately sized for use in a minimally invasive procedure and relatively simple in design to reduce possible points of failure. In addition, such mechanisms should provide an adequate range of motion to allow the end effector to be manipulated in a wide variety of positions.

For devices having opposing jaws, a significant amount of mechanical force must be applied by the end effector to effectively grasp and handle, seal, clamp, staple or cut tissue and vessels within a patient. For example, in a typical conventional device with opposing jaws, a cam slot pin is translated through cam slots in each of the jaws. The cam slots are typically linear and substantially parallel to the tissue contacting surface of each of the jaws. The jaws may be actuated by a drive element, such as a push-pull rod. In such a “push/pull” design, a single compression/tension element may be used to move the end effector component. Pulling (tension) is used to move the component in one direction, and pushing (compression) is used to move the component in the opposite direction. In typical devices, the compression force is used to actuate the end effector component in the direction that requires the highest force (e.g., closing the jaws).

The length of the cam slot defines the length of the gripping/actuation motion of a given surgical instrument. The cam slot pin is operatively coupled to a drive element (not shown), and rides through the cam slots upon actuation, transitioning the jaws between open and closed positions as they pivot around a distal pivot pin.

Because the cam slots are linear, the forces generated upon actuation of the surgical instrument vary depending on the angle between the jaws at the particular point in the actuation stroke. Therefore, the cam slots provide for a high mechanical advantage at the end of the gripping/actuation motion, e.g., when the grip angle is about 0 to about 20 degrees. However, when the grip angle is about 20 degrees or higher, a linear cam slot often provides insufficient force to the jaws to clamp, seal and/or grasp tissue, which can negatively impact the performance of the instrument.

The linear configuration throughout the length of the cam slots may also be overly sensitive to input forces, thereby providing undesired output forces. This sensitivity can provide for unwanted jaw force output change based on small amount of input change (such as extra friction in the drive train), increasing the chances of inaccurate force output during instrument use.

Accordingly, while the new telesurgical systems and devices have proven highly effective and advantageous, still further improvements would be desirable. In general, it would be desirable to provide surgical instruments that include an end effector with jaws capable of providing a sufficient gripping force throughout the actuation stroke (i.e., throughout the range of jaw angles between fully open and closed), thereby allowing the jaws to effectively grip, clamp, and/or seal tissue and vessels or varying size and diameter.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

The present disclosure provides a surgical instrument with an end effector having opposing jaws that open and close relative to each other. The jaws each include a cam slot for receiving a pin. Translation of the pin through the cam slots opens and closes the jaws. At least one of the cam slots is shaped such that at least one of the jaws applies a grip force that is substantially proportional to a force applied to the pin to translate the pin through at least one portion of the slots (i.e., the ratio between the input force and the resulting output force remains substantially the same as the pin travels through at least one portion of the slots). This design provides a constant mechanical advantage between the force applied to the pin and the force applied by the jaws to tissue held therebetween, thereby allowing a user (or a robotic system) to more easily regulate the forces applied to tissue by the jaws.

In addition, this design allows for a substantially constant grip force to be applied by the jaws regardless of the angle between the jaws. Therefore, the jaws may apply substantially the same amount of grip force against, for example, a larger vessel or tissue portion that requires the jaws to remain further open (e.g., greater than 20% of the fully open jaw configuration).

In one aspect, a surgical instrument comprises an elongate shaft and an end effector mounted on the elongate shaft. The end effector includes first and second jaws each having a cam slot for receiving a pin and being movable relative to each other between open and closed positions. At least one of the cam slots has a non-linear shape. The pin is positioned within the slots such that translation of the pin through the slots rotates the jaws between the open and closed positions.

In one embodiment, the non-linear slot has a proximal end and a distal end, and is curved from the proximal end to the distal end. The non-linear slot is shaped such that a grip force applied by at least one of the first and second jaws is substantially proportional to a force applied to the pin as the pin is translated from the proximal end to the distal end of the non-linear slot. Preferably, the non-linear slot is shaped such that the first and second jaws apply a substantially constant grip force therebetween as the pin is translated from the proximal end to the distal end of the slot. This provides a constant mechanical advantage between the force applied to the pin and the force applied by the jaws to tissue held therebetween, thereby allowing a user (or a robotic system) to more easily regulate the forces applied to tissue by the jaws. In addition, this design allows for a substantially constant grip force to be applied by the jaws regardless of the angle between the jaws.

In another embodiment, the non-linear slot is a compound slot that comprises a substantially linear proximal portion and a curved distal portion (or vice-versa). The distal portion of the compound slot is shaped such that the first and second jaws apply a substantially constant grip force therebetween as the pin is translated distally through the distal portion (i.e., the force applied by movement of the first jaw is substantially proportional to the force applied to the pin as the pin is translated distally through the proximal portion). The proximal portion of the compound slot is shaped to provide a non-constant grip force between the first and second jaws as the pin is translated through the proximal portion (i.e., the force applied by movement of the first jaw increases non-proportionally relative to the force applied to the pin as the pin is translated distally through said proximal portion).

In this embodiment, pulling the cam slot pin proximally causes the jaws to close and pushing the cam slot pin distally causes the jaws to open. Of course, it will be recognized that this configuration can be reversed (i.e., distal movement of the pin causes closure of the jaws). The curved distal portion of the slot provides a substantially constant mechanical advantage when the jaws are partially or substantially open. In this configuration, the jaws are typically used to perform tasks, such as tissue handling. This ensures that the jaws have sufficient gripping force against larger tissue or vessels when the jaws are partially or substantially open, and allows the user to more easily regulate the forces being applied to tissue grasped between the jaws.

The substantially linear proximal portion of the slot provides an elevated mechanical advantage as the pin travels through this portion (i.e., the jaws apply a stronger grip force as they close). In this configuration, the jaws are typically being used for sealing vessels. Elevating the mechanical advantage between the input force (i.e., the force applied to the pin) and output force (i.e., the forces applied by the jaws to tissue) enhances tissue/vessel compression and seal.

In another embodiment, both of the cam slots in the first and second jaws have curved distal portions that operate in combination to provide a constant mechanical advantage to the jaws. In yet another embodiment, the end effector may include multiple cam slot pins. For example, a proximal cam slot pin may translate through a curved distal cam slot in one of the jaws and a proximal cam slot pin translate through a substantially linear cam slot. The distal cam slot pin actuates the jaws for a first portion of the actuation stroke and the proximal cam slot pin actuates the jaws for a second portion of the actuation stroke.

In an exemplary embodiment, the first and second jaws define a first angle therebetween in the fully open position, and a second angle therebetween when the pin is located at a junction between the distal and proximal portions of the compound slot. The second angle is preferably about 50% or less the first angle, more preferably about 20% or less. Thus, the proximal portion of the slot corresponds with an angle of about 50% or less, preferably about 20% or less, of the total angle between the jaws in the fully open configuration. For example, when the jaws are at least 50% open (or at least 20% open in certain embodiments), the pin resides in the curved distal portion of the slot and the force applied by the jaws to tissue is substantially proportional to the force applied to the pin as the pin translates through the distal portion. When the jaws are less than 50% open (or less than 20% open in certain embodiments), the pin resides in the linear proximal portion of the slot and the force applied by the jaws to tissue is non-proportional to the force applied to the pin (i.e. elevated mechanical advantage) as the pin translates through the proximal portion.

In another aspect, an end effector for a surgical instrument comprises first and second jaws movable relative to each other between open and closed positions. The first jaw comprises a non-linear slot and the second jaw comprises a substantially linear slot. The end effector further includes a pin positioned within the linear and non-linear slots such that translation of the pin through the slots rotates the jaws between the open and closed positions.

In one embodiment, the non-linear slot has a proximal end and a distal end, and is curved from the proximal end to the distal end. The non-linear slot is shaped such that a force applied by movement of the first jaw is substantially proportional to a force applied to the pin as the pin is translated from the proximal end to the distal end of the non-linear slot. Preferably, the non-linear slot is shaped such that the first and second jaws apply a substantially constant grip force therebetween as the pin is translated from the proximal end to the distal end of the non-linear slot

In another embodiment, the non-linear slot comprises a compound slot with a curved distal portion and a substantially linear proximal portion. The curved distal portion of the compound slot is shaped such that a force applied by movement of the first jaw is substantially proportional to a force applied to the pin as the pin is translated distally through said distal portion. The linear proximal portion of the compound slot is shaped such that a force applied by movement of the first jaw increases non-proportionally relative to the force applied to the pin as the pin is translated distally through said proximal portion.

Preferably, the curved distal portion of the compound slot is shaped such that the first and second jaws apply a substantially constant grip force therebetween as the pin is translated distally through said distal portion. The linear proximal portion of the compound slot is shaped to provide a non-constant grip force between the first and second jaws as the pin is translated through said proximal portion.

In an exemplary embodiment, the first and second jaws define a first angle therebetween in the fully open position, and a second angle therebetween when the pin is located at a junction between the distal and proximal portions of the compound slot. The second angle is preferably about 50% or less the first angle, more preferably about 20% or less.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description serve to explain the principles of the disclosure.

FIG. 1 is a side view of the distal end portion of a surgical instrument in accordance with an illustrative embodiment of this disclosure with the jaws of the end effector in the closed position;

FIG. 2 is a perspective view of the distal end portion of the surgical instrument of FIG. 1;

FIG. 3 is a side view of the distal end portion of the surgical instrument of FIG. 1 with the jaws of the end effector in the open position;

FIG. 4 is a partial cross-sectional view of an end effector of a surgical instrument in accordance with the present disclosure;

FIG. 5 is a partial cross-sectional view of another embodiment of an end effector of a surgical instrument in accordance with the present disclosure;

FIG. 6A is a front elevation, diagrammatic view of an exemplary patient side cart of a teleoperated surgical system;

FIG. 6B is a front elevation, diagrammatic view of an exemplary surgeon's console of a teleoperated surgical system;

FIG. 6C is a front elevation, diagrammatic view of an exemplary auxiliary control/vision cart of a teleoperated surgical system;

FIG. 7 is a perspective view of an illustrative teleoperated surgical instrument usable with an end effector of the present disclosure; and

FIG. 8 illustrates a perspective view of an illustrative surgical instrument with an end effector of the present disclosure.

### DETAILED DESCRIPTION

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

While the following disclosure is presented with respect to an end effector for a surgical instrument having two opposing jaws for clamping and/or sealing tissue, it should be understood that the features of the presently described invention may be readily adapted for use in any type of surgical clamping, cutting, stapling or sealing instrument. For example, the presently described end effector may be employed in an surgical stapling instrument, such as any of those instruments described in commonly-assigned, co-pending U.S. Provisional Patent Application Nos. 62/947,307, 62/947,263 and 62/961,504; U.S. patents application Ser. Nos. 16/205,128, 16/678,405 and 16/904,482; and International Patent Nos. PCT/US2019/107646, PCT/US2019/019501, PCT/US2019/062344, PCT/US2019/064861, PCT/US2019/062768, PCT/2020/025655, PCT/US2020/056979, PCT/2019/066513, PCT/US2020/020672 and PCT/US2019/066530 and PCT/US2020/033481, the complete disclosures of which are incorporated by reference herein in their entirety for all purposes as if copied and pasted herein.

An end effector for a surgical instrument in accordance with embodiments of the present disclosure includes first and second jaws configured to grasp tissue therebetween. In certain embodiments, at least one of the jaws includes a sealing electrode on a tissue contacting surface thereof for applying energy to tissue grasped between the jaws, and a cutting electrode to dissect tissue previously (or simultaneously) sealed by the sealing electrodes. The cutting electrode may extend beyond the tissue contacting surface of the jaw and into a slot on the other jaw. In embodiments, the cutting electrode is part of an electrode assembly that includes structure (e.g., a heat stake post) to permanently mount the electrode assembly to the jaw. In embodiments, separate conductor wires supply electrosurgical energy to the sealing and cutting electrodes.

Surgical instruments of the present disclosure are adapted to be used with a robotic system for treating tissue with electrosurgical energy (e.g., cutting, sealing, ablating, etc.). The surgical instruments will generally include an actuation mechanism that controls the orientation and movement of the end effector. The actuation mechanism will typically be controlled by a robotic manipulator assembly that is controlled remotely by a user. For example, in one configuration, the actuation mechanism will be manipulated by the robotic manipulator assembly to move the jaws of the end effector between an open position and a closed position. In the closed position, the end effector will contact the electrodes against the tissue to cauterize and/or sever the engaged tissue. While described herein with respect to an instrument configured for use with a robotic surgical system, it should be understood that the end effectors and other structures of the surgical instruments described herein may be incorporated into manually actuated instruments, electro-mechanical powered instruments, or instruments actuated in any other way.

The electrodes disposed on the end effector are contacted against the tissue so that current will flow from one electrode to the other electrode through the engaged tissue. In some configurations, the electrodes will both be disposed on the same end effector. When the end effector is in the closed position, the electrodes will be offset and spaced from each other such that delivery of a high frequency electrical energy will flow through the tissue between the electrodes without shorting the electrodes. Even if no tissue is between the end effector, there will typically be a gap between the electrodes. When the end effector is in the closed position the spacing between the negative and positive electrode will generally be from about 0.01 and 0.10 inches, and in embodiments from about 0.010 inches and 0.025 inches. It should be appreciated however, that the spacing of the electrodes will vary depending on the area, volume, width, material of the electrodes, and the like. Similarly, electrode spacing and geometry may be adjusted to accommodate target tissue properties such as tissue thickness and impedance.

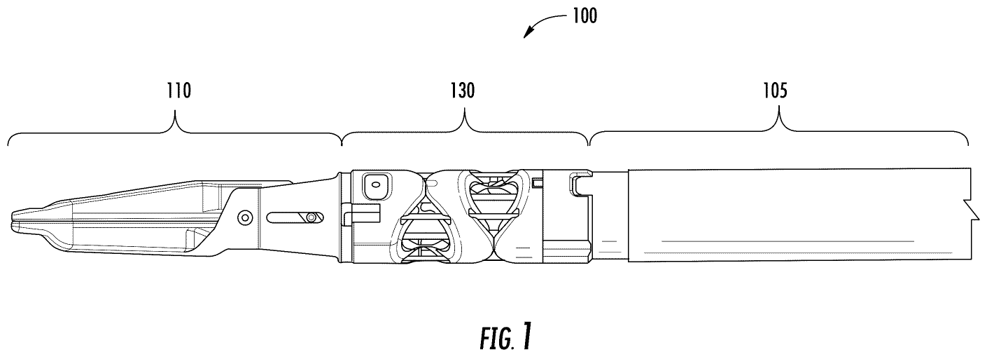

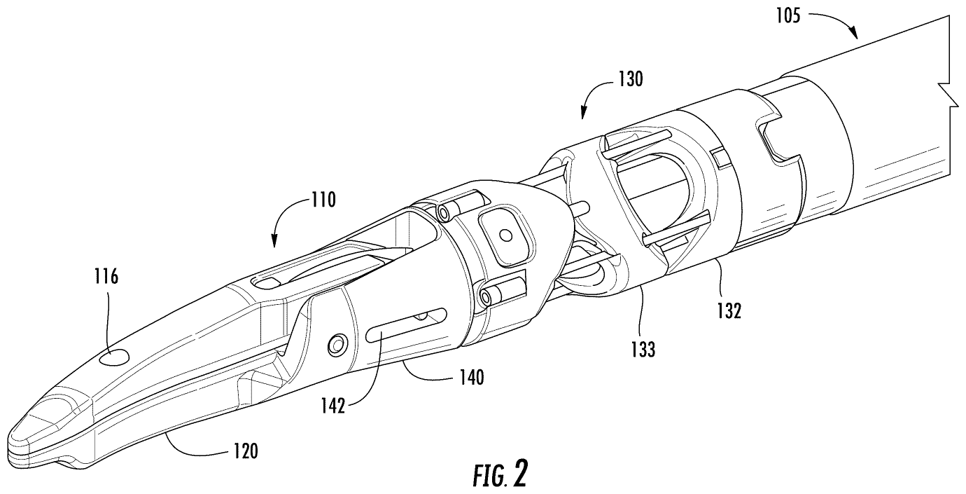

FIGS. 1 and 2 show the distal end portion of a surgical instrument 100 in accordance with an illustrative embodiment of the present disclosure. Surgical instrument 100 includes an end effector 110, an articulation mechanism 130, and an elongated shaft 105. The proximal end portion of elongate shaft 105 is operatively connected to an actuation mechanism (not shown), although as those skilled in the art reading this disclosure will appreciate, components of the actuation mechanism may extend into, and/or pass through elongated shaft 105 and/or articulation mechanism 130.

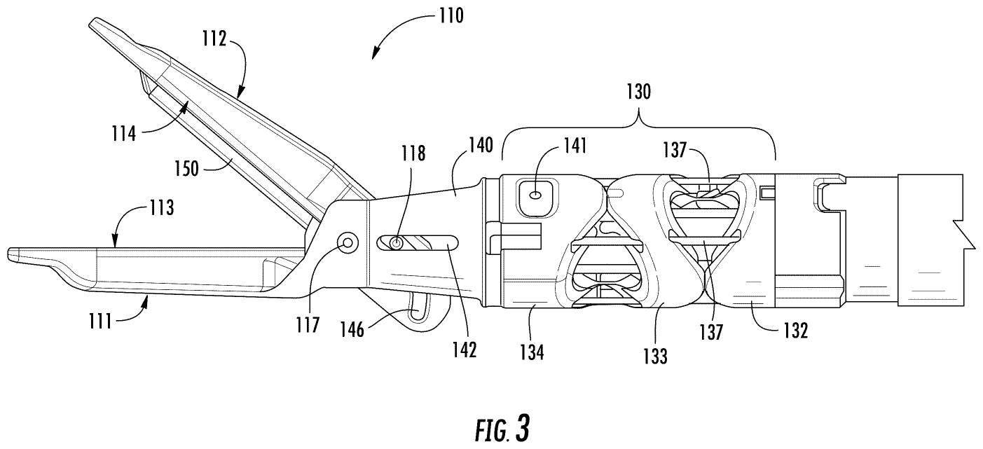

With reference now to FIG. 3, end effector 110 includes a first jaw 111, and a second jaw 112 configured to move between an open position (as shown in FIG. 3) where the jaws are spaced apart from one another and a closed position (shown in FIGS. 1 and 2) where tissue contacting surfaces 113, 114 cooperate to grasp tissue therebetween. In certain embodiments, first jaw 11 is a movable jaw configured to move from an open position to a closed position relative to second jaw 112. In other embodiments, first jaw 111 is a movable jaw configured to move between open and closed positions relative to second jaw 112. In still other embodiments, both jaws 111, 112 are movable relative to each other. In the exemplary embodiment shown, first jaw 111 is stationary and second jaw 112 is movable relative to first jaw 112 to pivot jaws 111, 112 between a fully open position, wherein the jaws define a desired angle between each other, to a substantially closed position, wherein the jaws are substantially parallel with each other.

Stationary jaw 111 and movable jaw 112 further include sealing electrodes (not shown) on tissue contacting surfaces 113, 114 for coagulating tissue grasped between jaws 111, 112. A cutting electrode 150 extends along a portion of movable jaw 112 and beyond tissue-contacting surface 114 for delivering high frequency electrical energy to sever grasped tissue. Stationary jaw 111 includes a central slot (not shown) into which cutting electrode 150 may extend when the jaws 111, 112 are in the closed position. Separate electrical pathways are provided to feed electrical current to each of the sealing electrode(s) on stationary jaw 111, the sealing electrode(s) on movable jaw 112, and the cutting electrode 150 on movable jaw 112.

Stationary jaw 111 includes a clevis 140 at a proximal portion thereof as seen in FIG. 2. A longitudinal slot 142 is formed on clevis 140. As those skilled in the art will appreciate, the opposing side of end effector 110 (not seen in FIG. 2) is substantially symmetrical to and may include the same features as the side of end effector 110 shown in FIG. 2. Slot 142 is substantially parallel to the tissue contacting surface 113 of stationary jaw 111. Movable jaw 112 includes a cam slot 146 (see FIG. 3). Cam slot 146 may be straight, curved, or may include a compound configuration in which a portion is straight and a portion is curved to achieve a desired mechanical advantage along different portions of a jaw opening and closing mechanism. A cam pin 118 travels along longitudinal slot 142 and within cam slot 146 such that proximal and distal translation of pin 118 pivots movable jaw 112 between the open and closed position.

Movement of jaws 111, 112 between the open and closed position is achieved by an actuation mechanism including a drive element (not shown). In such a “push/pull” design, a compression/tension element may be used to move the end effector component. Pulling (tension) is used to move the component in one direction, and pushing (compression) is used to move the component in the opposite direction. In some implementations, the tension force (pulling) is used to actuate the end effector component in the direction that requires the highest force (e.g., closing jaws). The length of slot 142 defines the length of the gripping/actuation motion of a given surgical instrument. Cam pin 118 is operatively coupled to a drive element (not shown), and rides through slots 142 and 146 upon actuation, transitioning jaws 111, 112 between the open and closed positions as they pivot around a pivot pin 117. In the exemplary embodiment, as cam pin 118 is pulled in the proximal direction, jaws 111, 112 pivot towards the closed position to grasp tissue.

Surgical instruments in accordance with this disclosure may employ drive cables that are used in conjunction with a system of motors and pulleys. Powered surgical systems, including robotic surgical systems that utilize drive cables connected to a system of motors and pulleys for various functions including opening and closing of jaws, as well as for movement and actuation of end effectors are well known. Further details of known drive cable surgical systems are described, for example, in U.S. Pat. No. 7,666,191 and U.S. Pat No. 9,050,119 both of which are hereby incorporated herein by reference in their entireties.

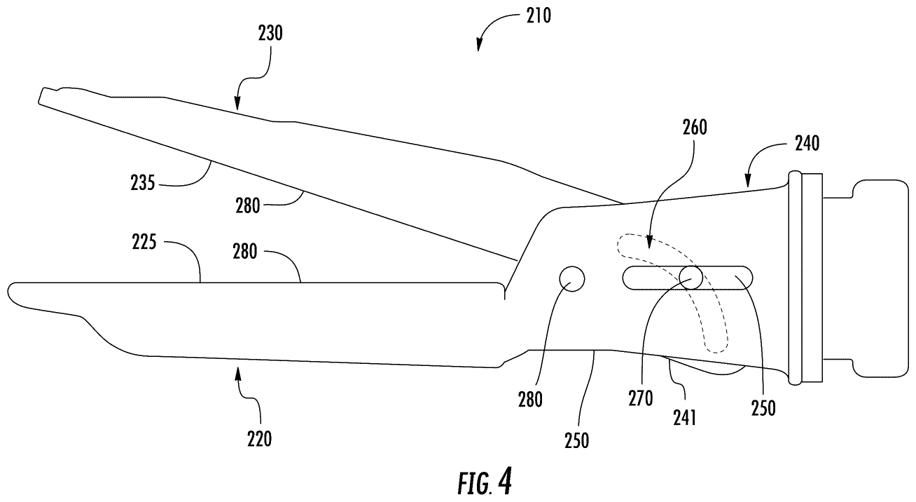

FIG. 3 shows a side view of end effector 110 and articulation mechanism 130 (e.g., wrist assembly 130) positioned between clevis 140 and elongated shaft 105. Wrist assembly 130 may provide a desired amount of motion, such as +/-90 degrees in a pitch or yaw direction. Wrist assembly 130 includes a proximal link 132, a middle link 133, and a distal link 134 that collectively determine the kinematic pitch and yaw motion of the wrist assembly 130. As shown, the interface between the proximal link 132 and the middle link 133 defines a joint that affects yaw movement of wrist assembly 130. The interface between the distal link 134 and the middle link 133 defines a joint that determines pitch movement of wrist assembly 130. However, in an alternative embodiment of a wrist assembly, this relationship can be reversed such that wrist assembly 130 pitches between proximal link 132 and middle link 133 and yaws between distal link 134 and middle link 133. Distal link 134 includes distal link halves that may be welded or otherwise secured to each other and rigidly attached to and provide mechanical connection and electrical isolation of wrist assembly 130 to clevis 140 of stationary jaw 111 via post 141. (See FIGS. 3 and 4.) Cables 137 are drivingly coupled with the wrist assembly 130 and actuated to impart motion to wrist assembly 130. Differential movement of cables 137 can be used to actuate wrist assembly 130 to pitch and yaw at various angles. Additional details of articulation mechanisms usable with the embodiments disclosed herein are disclosed in Int'l. Pub. No. WO 2015/127250A1 and U.S. Publication No. 2017/0215977 A1, the entire disclosure of each of which is incorporated by reference herein.

Referring now to FIG. 4, an end effector 210 for a surgical instrument, such as the illustrative surgical instrument shown in FIGS. 1-3, will now be described. As shown, end effector 210 includes first and second jaws 220, 230 which may be attached to a surgical instrument via a clevis 240. Clevis 240 further includes an opening for receiving a pivot pin 280 defining a pivot axis around which jaws 220, 230 pivot, as described in more detail below. A more complete description of a suitable clevis 240 for use with the present invention may be found in commonly-assigned, co-pending provisional patent application Nos. 62,783,444, filed Dec. 21, 2018; 62,783,481, filed Dec. 21, 2018; 62,783,460, filed Dec. 21, 2018; 62,747,912, filed Oct. 19, 2018; and 62,783,429, filed Dec. 21, 2018, the complete disclosures of which are hereby incorporated by reference in their entirety for all purposes. Of course, it will be recognized by those skilled in the art that other coupling mechanisms known by those skilled in the art may be used with the present invention to attach the jaws 220, 230 to the proximal portion of a surgical instrument.

In certain embodiments, first jaw 220 is a movable jaw configured to move from an open position to a closed position relative to second jaw 230. In other embodiments, first jaw 220 is a movable jaw configured to move between open and closed positions relative to second jaw 230. In still other embodiments, both jaws 220, 230 are movable relative to each other. In the exemplary embodiment shown, first jaw 220 is stationary and second jaw 230 is movable relative to first jaw 220 to pivot jaws 220, 230 between a fully open position, wherein the jaws define a desired angle between each other, to a substantially closed position, wherein the jaws are substantially parallel with each other.

In certain embodiments, end effector 210 may be articulated in multiple directions by an articulation mechanism, such as a wrist (not shown), although other articulation mechanisms are contemplated. Other articulation mechanisms known by those skilled in the art may substitute for wrist 160 as shown for example in U.S. Publication. No. 2015/0250530 the entire disclosure of which is hereby incorporated by reference in its entirety for all purposes.

According to one embodiment of the present disclosure, first jaw 220 comprise a substantially linear cam slot 250, and second jaw 230 comprises a non-linear cam slot 260. A cam slot pin 270 is disposed within cam slots 250, 260 and configured to translate distally and proximally therethrough. Distal translation of cam slot pin 270 causes second jaw 230 to close relative to first jaw 220 and proximal translation of cam slot pin 270 causes the jaws 220, 230 to open.

In an exemplary embodiment, cam slot 260 is curved and preferably shaped such that second jaw 230 applies a grip force against first jaw 220 that is substantially proportional to a force applied to cam slot pin 270 to translate pin 270 through slots 250, 260 (i.e., the ratio between the force input and the resulting force output remains substantially the same as pin 270 travels through the entire length of slots 250, 260). This design provides a constant mechanical advantage between the force applied to cam slot pin 270 and the force applied by jaws 220, 230 to tissue held therebetween, thereby allowing a user (or a robotic system) to more easily regulate the forces applied to tissue by jaws 220, 230.

In addition, this design allows for a substantially constant grip force to be applied by jaws 220, 230 regardless of the angle between jaws 220, 230. Therefore, at least in certain embodiments, 220, 230 jaws may apply substantially the same amount of grip force against, for example, a larger vessel or tissue portion that requires jaws 220, 230 to remain further open (e.g., from 100% of the fully open position down to 20% of the fully open position) than the force jaws 220, 230 would apply in a more closed position (i.e., less than 20% of the fully open position).

In an exemplary embodiment, non-linear slot 260 is configured and dimensioned to provide a constant mechanical advantage to end effector 210 as cam slot pin 270 moves throughout the length of slot 260 and the gripping/actuation motion. Applicant has discovered a critical profile for cam slot 260 that will provide this constant mechanical advantage throughout substantially the entire range of motion of jaws 220, 230. Assuming there is relatively low friction compared to driving forces, (which can be provided by the surface finish in cam slots 250, 260 and pin 270 travelling therethrough), and that the forces are transferred to the central axis of pin 270, the profile of cam slot 260 to provide for constant mechanical advantage may be determined using the following equation:

$$R ( θ ) = \frac{( a - b )}{λ} θ + b$$

wherein R is the cam slot profile as a function of jaw angle θ, a is the distance between distal pivot pin 280 and cam slot pin 270 when the jaws are in a fully open configuration, b is the distance between distal pivot pin 280 and cam slot pin 270 when the jaws are fully closed, λ is the maximum jaw angle when the jaws are 100% open, and θ is the instantaneous jaw angle having a range from 0 to λ.

A cam slot profile derived from this equation allows for greater control over forces exerted by the jaws during the entire ranging of motion of jaw opening and closing, as the force applied by the jaws will be a constant multiple of the force applied to the pin by the drive mechanism. For the majority of an instrument's range of motion, tissue handling is a high priority, and the foregoing configuration of cam slot 260 provides for constant mechanical advantage that allows a user to more easily regulate the forces being applied while grasping tissue.

Of course, it will be recognized by those of skill in the art that the present disclosure is not limited to the above embodiment. For example, cam slot 250 may be curved and cam slot 260 substantially linear. In this embodiment, cam slot 250 provides the substantially constant mechanical advantage to jaws 220, 230. In another configuration, both cam slots 250, 260 may be curved and shaped in combination to provide a substantially constant mechanical advantage to jaws 220, 230.

In certain embodiments, end effector 210 may further include electrodes 280 on one or both of the jaws in order to function as an electrosurgical instrument. In bipolar embodiments, electrodes 280 comprise tissue contacting surfaces 225, 235 on each of the jaws 220, 230. Electrodes 280 are then connected to output electrodes of electrical generators such that the opposing jaws are charged to different electrical potentials. Organic tissue, being electrically conductive, thereby allows for the two electrodes to apply electrical current through the grasped tissue in the closed position to heat tissue or blood vessels to cause coagulation or cauterization. For additional details on general aspects of electrosurgical instruments such as those described herein, see, e.g., U.S. Pat. No. 5,674,220, the entire disclosure of which is incorporated herein by reference for all purposes.

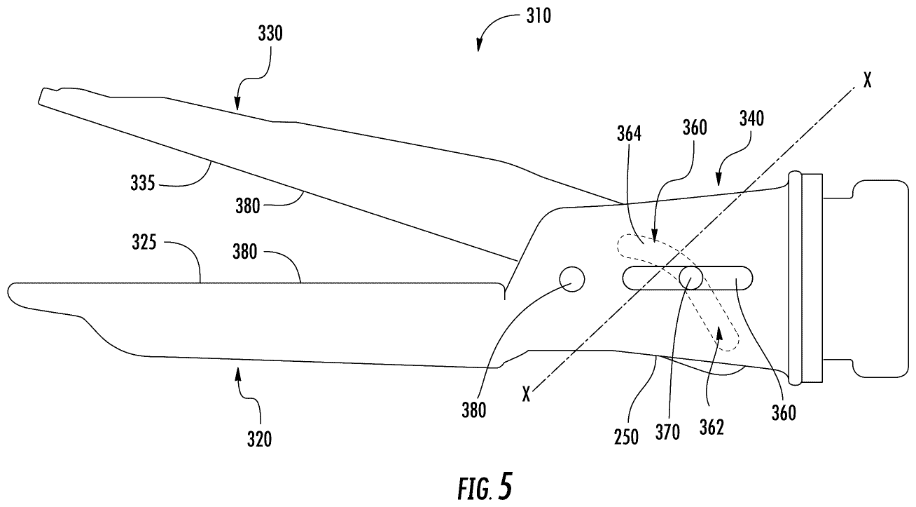

FIG. 5 illustrates another embodiment of an end effector 310 according to the present disclosure. Similar to the previous embodiment, end effector 310 includes first and second jaws 320, 330 which may be attached to a surgical instrument via a clevis 340. Clevis 340 further includes an opening for receiving a pivot pin 380 defining a pivot axis around which jaws 320, 330 pivot, as described in more detail below. In certain embodiments, first jaw 320 is a movable jaw configured to move from an open position to a closed position relative to second jaw 330. In other embodiments, first jaw 320 is a movable jaw configured to move between open and closed positions relative to second jaw 330. In still other embodiments, both jaws 320, 330 are movable relative to each other. In the exemplary embodiment shown, first jaw 320 is stationary and second jaw 330 is movable relative to first jaw 320 to pivot jaws 320, 330 between a fully open position, wherein the jaws define a desired angle between each other, to a substantially closed position, wherein the jaws are substantially parallel with each other.

First jaw 320 comprise a substantially linear cam slot 350, and second jaw 330 comprises a compound cam slot 360. A cam slot pin 370 is disposed within cam slots 350, 360 and configured to translate distally and proximally therethrough. Proximal translation of cam slot pin 370 causes second jaw 330 to close relative to first jaw 320 and distal translation of cam slot pin 370 causes the jaws 320, 330 to open.

Compound cam slot 360 comprises a non-linear distal portion 364 and a substantially linear proximal portion 362 (the junction between proximal portion 364 and distal portion 362 is indicated by dotted line X-X). Distal portion 364 is shaped such that jaws 320, 330 apply a substantially constant grip force therebetween as cam slot pin 370 is translated proximally through distal portion 364 (i.e., the force applied by movement of first jaw 330 is substantially proportional to the force applied to cam slot pin 370 as pin 370 is translated proximally through distal portion 364). Proximal portion 362 of compound slot 360 is shaped to provide a non-constant grip force between jaws 320, 330 as cam slot pin 370 is translated through proximal portion 362 (i.e., the force applied by movement of first jaw 330 increases non-proportionally relative to the force applied to can slot pin 370 as pin 370 is translated distally through proximal portion 362).

The curved distal portion 364 of compound slot 360 provides a substantially constant mechanical advantage when jaws 320, 330 are partially or substantially open. In this configuration, jaws 320, 330 are typically used to perform tasks, such as tissue handling. This allows the user to more easily regulate the forces being applied to tissue grasped between jaws 320, 330. In an exemplary embodiment, distal portion 364 has a profile to provide for constant mechanical advantage similar to, or the same as, the profile described above with respect to cam slot 260 in FIG. 4.

The substantially linear proximal portion 362 of compound slot 360 provides an elevated mechanical advantage as cam slot pin 370 travels through proximal portion 362 (i.e., jaws 320, 330 apply a stronger grip force as they close). In this configuration, jaws 320, 330 are typically being used for sealing vessels. Elevating the mechanical advantage between the input force (i.e., the force applied to pin 370) and output force (i.e., the forces applied by jaws 320, 330 to tissue) enhances tissue/vessel compression and seal.

Of course, it will be recognized that other configurations are possible. For example, cam slot 350 may be a compound slot while slot 360 is substantially linear. Alternatively, both cam slots 350, 360 may have curved proximal portions that operate in combination to provide a constant mechanical advantage to jaws 320, 330. In yet another embodiment, end effector 310 may include multiple cam slot pins. For example, a proximal cam slot pin may translate through a curved proximal cam slot in one of the jaws and a distal cam slot pin translate through a substantially linear cam slot. The proximal cam slot pin actuates jaws 320, 330 for a first portion of the actuation stroke and the distal cam slot pin actuates jaws 320, 330 for a second portion of the actuation stroke.

In an exemplary embodiment, first and second jaws 320, 330 define a first angle therebetween in the fully open position, and a second angle therebetween when cam slot pin 370 is located at a junction between distal and proximal portions 362, 364 of compound slot 360. The second angle is preferably about 50% or less the first angle, more preferably about 20% or less. Thus, proximal portion 362 of compound slot 360 corresponds with an angle of about 50% or less, preferably about 20% or less, of the total angle between jaws 320, 330 in the fully open configuration. For example, when jaws 320, 330 are at least 50% open (or at least 20% open in certain embodiments), cam slot pin 370 resides in the curved distal portion 364 of compound slot 360 and the force applied by jaws 320, 330 to tissue is substantially proportional to the force applied to cam slot pin 370 as pin 370 translates through distal portion 364. When jaws 320, 330 are less than 50% open (or less than 20% open in certain embodiments), cam slot pin 370 resides in the substantially linear proximal portion 362 of compound slot 360 and the force applied by jaws 320, 330 to tissue is non-proportional to the force applied to pin 370 (i.e. elevated mechanical advantage) as pin 370 translates through proximal portion 362.

In an exemplary embodiment, distal portion 364 is actively engaged by cam slot pin 370 within cam slot 360 when the jaws are between about 20% to about 100% open and proximal portion 362 is actively engaged by cam slot pin 370 within cam slot 360 when the jaws are between about 0% to about 20% open. Cam slot 360 has a point of inflection along an axis X-X, where cam slot 360 transitions from providing a constant mechanical advantage, to providing a non-constant mechanical advantage as cam slot pin 370 is translated from the distal portion 364 to the proximal portion 362.

The resulting compound cam slot 360 created by the combination of proximal portion 362 and distal portion 364 allows a user to have the control and benefits of constant mechanical force throughout a relatively large portion of the actuation stroke. Additionally, as the actuation stroke nears completion, a user benefits from the proximal portion 362 of the cam slot which is configured to provide a higher mechanical advantage to ensure that sufficient clamping force is achieved before the instrument's function is carried out, such as sealing, stapling, or other useful functions. For sealing, a compound cam slot in accordance with this disclosure may be configured to ensure that a user achieves operating pressures of about 3 kg/cm² to about 16 kg/cm² to effect a proper and effective tissue seal.

End effector 310 may further include an electrodes 390 on one or both of the jaws in order to function as an electrosurgical instrument. In bipolar embodiments, electrodes 390 may comprise tissue contacting surfaces 325,335 on each of the jaws 320,330.

The end effectors in accordance with the presently described embodiments may be readily adapted for use in any type of surgical clamping, cutting, and/or sealing instruments. For example, features of the present surgical instruments may be employed to treat tissue with electrosurgical energy (e.g., cutting, sealing, ablating, etc.). The surgical instrument including the present end effectors may be a minimally invasive (e.g., laparoscopic) instrument or an instrument used for open surgery.

Additionally, the features of the presently described end effectors for surgical stapling instruments may be readily adapted for use in surgical instruments that are activated using any technique within the purview of those skilled in the art, such as, for example, manually activated surgical instruments, powered surgical instruments (e.g., electro-mechanically powered instruments), robotic surgical instruments, and the like.

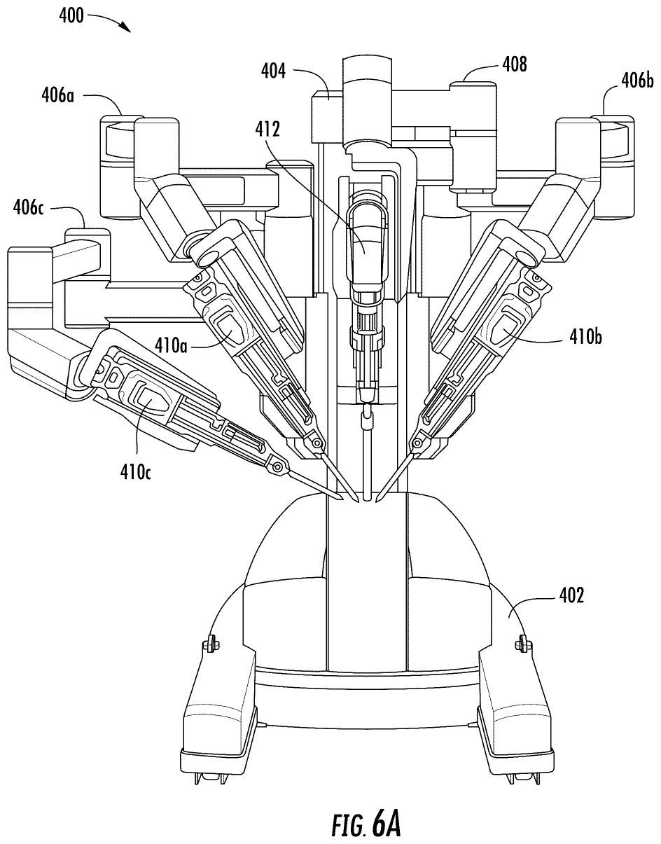

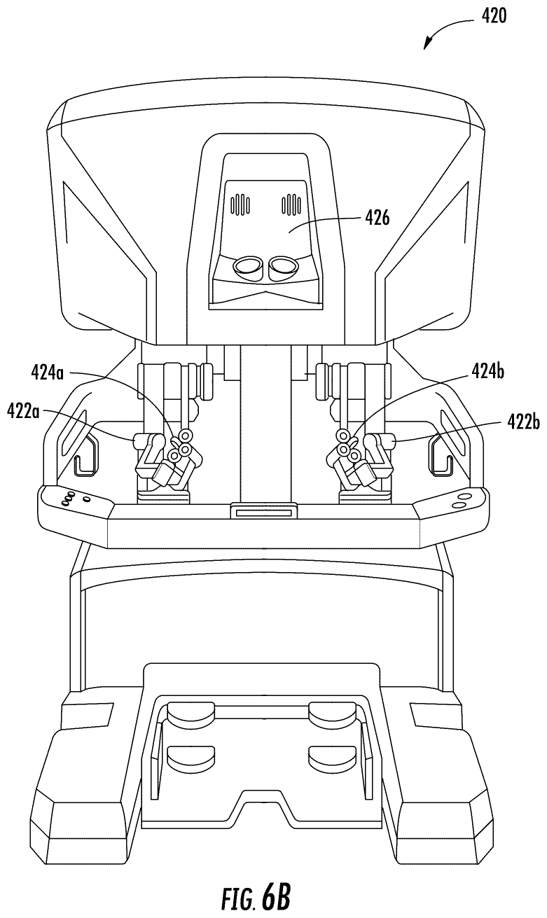

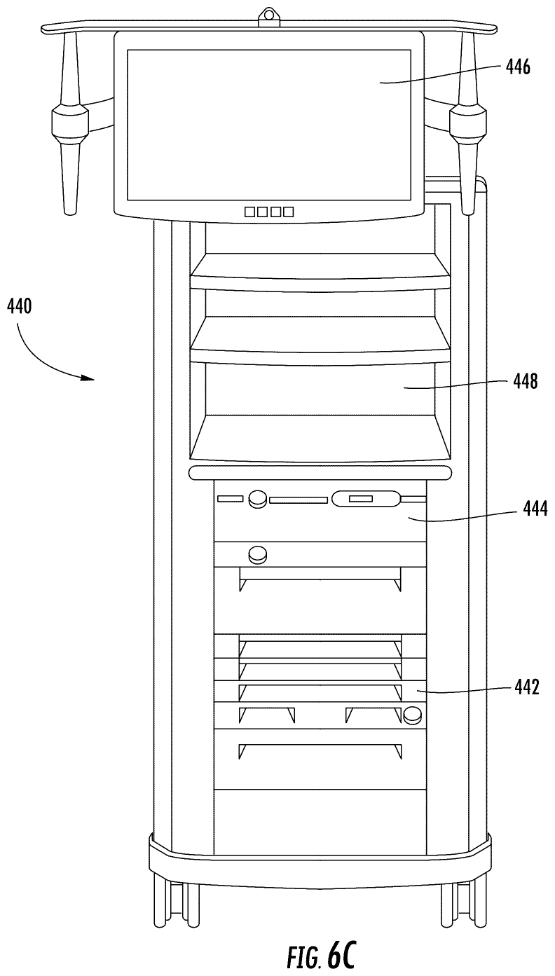

In certain embodiments, the end effectors described above in accordance with this disclosure may be used with surgical instruments incorporated into a robotic surgical system. FIGS. 6A, 6B, and 6C are front elevation views of three exemplary embodiments of main components of a teleoperated surgical system for minimally invasive surgery that may be used in combination with end effectors of the present disclosure. These three components are interconnected so as to allow a surgeon, for example, with the assistance of a surgical team, to perform diagnostic and corrective surgical procedures on a patient. In an exemplary embodiment, a teleoperated surgical system in accordance with the present disclosure may be embodied as a da Vinci® surgical system commercialized by Intuitive Surgical, Inc. of Sunnyvale, Calif. Also, for a further explanation of a teleoperated surgical system, including a patient side cart, surgeon's console, and auxiliary control/vision cart, with which the present disclosure may be implemented, reference is made to U.S. Patent App. Pub. No. 2011/0071542A1, which is incorporated by reference in its entirety herein. However, the present disclosure is not limited to any particular surgical system, and one having ordinary skill in the art reading this disclosure would appreciate that the disclosure herein may be applied in a variety of surgical applications, including other teleoperated surgical systems.

FIG. 6A is a front elevation view of an exemplary embodiment of a patient side cart 100 of a teleoperated surgical system. The patient side cart 400 includes a base 402 that rests on the floor, a support tower 404 mounted on the base 402, and one or more manipulator arms mounted on the support tower 404 and that support surgical instruments and/or vision instruments (e.g., a stereoscopic endoscope). As shown in FIG. 6A, manipulator arms 406a, 406b are arms that support, and transmit forces to manipulate, the surgical instruments used to grasp and move tissue, and arm 408 is a camera arm that supports and moves the endoscope. FIG. 6A also shows a third manipulator arm 406c that is supported on the back side of support tower 404 and that is positionable to either the left or right side of the patient side cart as desired to conduct a surgical procedure.

Interchangeable surgical instruments 410a, 410b, 410c can be installed on

the manipulator arms 406a, 406b, 406c, and an endoscope 412 can be installed on the camera arm 108. Those of ordinary skill in the art reading this disclosure will appreciate that the arms that support the instruments and the camera may also be supported by a base platform (fixed or moveable) mounted to a ceiling or wall, or in some instances to another piece of equipment in the operating room (e.g., the operating table). Likewise, they will appreciate that two or more separate bases may be used (e.g., one base supporting each arm).

Control of the robotic surgical system, including control of the surgical instruments, may be effectuated in a variety of ways, depending on the degree of control desired, the size of the surgical assembly, and other factors. In some embodiments, the control system includes one or more manually operated input devices, such as a joystick, an exoskeletal glove, pincher or grasper assemblies, buttons, pedals, or the like. These input devices control servo motors which, in turn, control the articulation of the surgical assembly. The forces generated by the servo motors are transferred via drivetrain mechanisms, which transmit the forces from the servo motors generated outside the patient's body through an intermediate portion of the elongate surgical instrument 110 to a portion of the surgical instrument inside the patient's body distal from the servo motor.

FIG. 6B is a front elevation view of an exemplary surgeon's console 420 of a teleoperated surgical system for controlling the insertion and articulation of surgical instruments 410. The surgeon or other system operator manipulates input devices by moving and repositioning input devices within console 420. As illustrated in the exemplary embodiment of FIG. 6B, the surgeon's console is equipped with master controllers or master input devices. As illustrated in FIG. 6B, master input devices may include left and right multiple degree-of-freedom (DOF) master tool manipulators (MTM's) 422a, 422b, which are kinematic chains that are used to control the surgical tools (which include the endoscope and various cannulas mounted on arms 406, 408 of the patient side cart 400). Each MTM may include an area for surgeon or operator input. For example, as shown in FIG. 6B, each MTM 422a, 422b may include a pincher assembly 424a, 424b. The surgeon grasps a pincher assembly 424a, 424b on each MTM 422a, 422b, typically with the thumb and forefinger, and can move the pincher assembly to various positions and orientations. When a tool control mode is selected, each MTM 422 is coupled to control a corresponding manipulator arm 406 for the patient side cart 400, as those of ordinary skill in the art are familiar. The pincher assembly is typically used to operate a surgical end effector (e.g., scissors, grasping retractor, needle driver, hook, forceps, spatula, etc.) at the distal end of an instrument 410.

The surgeon's console 420 also can include an image display system 426. In an exemplary embodiment, the image display is a stereoscopic display wherein left side and right side images captured by the stereoscopic endoscope 412 are output on corresponding left and right displays, which the surgeon perceives as a three-dimensional image on display system 426.

The surgeon's console 420 is typically located in the same operating room as the patient side cart 400, although it is positioned so that the surgeon operating the console may be outside the sterile field. One or more assistants may assist the surgeon by working within the sterile surgical field (e.g., to change tools on the patient side cart, to perform manual retraction, etc.). Accordingly, the surgeon may operate remote from the sterile field, and so the console may be located in a separate room or building from the operating room. In some implementations, two consoles 420 (either co-located or remote from one another) may be networked together so that two surgeons can simultaneously view and control tools at the surgical site.

For additional details on the construction and operation of general aspects of a teleoperated surgical system such as described herein, see, e.g., U.S. Pat. No. 6,493,608 and U.S. Pat. No. 6,671,581, the entire disclosure of each of which is incorporated herein by reference.

As shown in FIG. 6C, the auxiliary control/vision cart 440 includes an optional display 446 (e.g., a touchscreen monitor), which may be mounted elsewhere, such as on the patient side cart 400. The auxiliary control/vision cart 440 further includes space 448 for optional auxiliary surgical equipment, such as electrosurgical units, insufflators, and/or other flux supply and control units. The patient side cart 400 (FIG. 6A) and the surgeon's console 420 (FIG. 6B) are coupled via optical fiber communications links to the auxiliary control/vision cart 440 so that the three components together act as a single teleoperated minimally invasive surgical system that provides an intuitive telepresence for the surgeon.

In accordance with various exemplary embodiments, the present disclosure contemplates controlling a surgical instrument such that a gripping force applied by an end effector of the instrument is substantially linear throughout a range of motion of the end effector for a given force applied to a push-pull (drive) rod of the instrument to actuate the end effector.

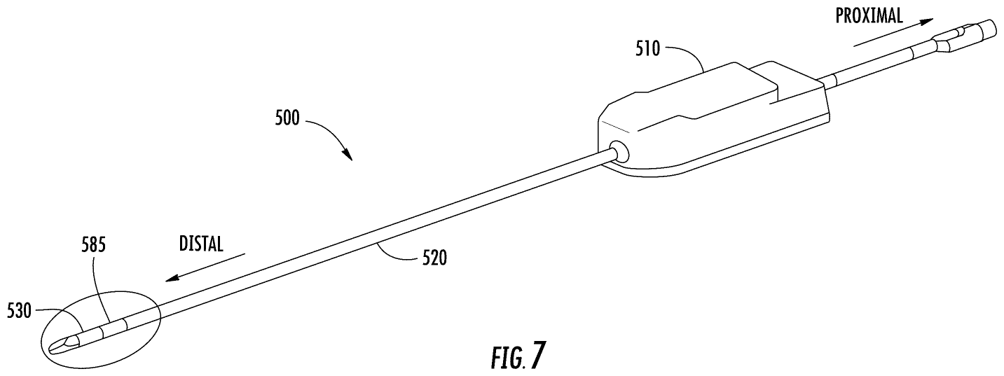

With reference to FIG. 7, an exemplary embodiment of a teleoperated surgical instrument 500 that may support a previously described end effector of the present disclosure is depicted. As shown in FIG. 7, surgical instrument 500 generally includes a housing 510 at its proximal end. Housing 510 may include an instrument memory or storage device (not shown). The memory can perform a number of functions when the instrument is loaded on the manipulator arm 406. For example, the memory can provide a signal verifying that the instrument is compatible with that particular surgical system. Additionally, the memory may identify the instrument and end effector type (whether it is a scalpel, a needle grasper, jaws, scissors, a clip applier, an electrocautery blade, or the like) to the surgical system so that the system can reconfigure its programming to take full advantage of the instrument's specialized capabilities. As further discussed below, the memory may include specifics on the architecture of the instrument, and include particular values that should be employed in control algorithms, such as tool compliance and gain values.

Housing 510 also may include a force/torque drive transmission mechanism (not shown) for receiving output from motors of the manipulator arm 406, the force/torque drive transmission mechanism transmitting the output from the motors to an end effector 530 of the instrument through an instrument shaft 520 mounted to the transmission mechanism. Exemplary surgical robotic instruments, instrument/manipulator arm interface structures, and data transfer between the instruments and servomechanism is more fully described in U.S. Pat. No. 6,331,181, the full disclosure of which is incorporated herein by reference.

Surgical instrument 500 comprises an end effector 530 disposed at the distal end of an elongate shaft 520 and may be connected thereto by a clevis 585 that supports and mounts end effector 530 relative to instrument shaft 520. As embodied herein, shaft 520 may be a relatively flexible structure that can bend and curve. Alternatively, shaft 520 may be a relatively rigid structure that does not permit traversing through curved structures. Optionally, in some embodiments, instrument 500 also can include a multi-DOF articulable wrist structure (not shown) that supports end effector 530 and permits multi-DOF movement of the end effector in arbitrary pitch and yaw. Those having ordinary skill in the art are familiar with a variety of wrist structures used to permit multi-DOF movement of a surgical instrument end effector.

For additional details on robotic surgical systems, see, e.g., commonly owned U.S. Pat. No. 6,493,608 “Aspects of a Control System of a Minimally Invasive Surgical Apparatus,” and commonly owned U.S. Pat. No. 6,671,581 “Camera Referenced Control in a Minimally Invasive Surgical Apparatus,” which are hereby incorporated herein by reference in their entirety for all purposes. A more complete description of illustrative robotic surgical systems for use with the present invention can be found in commonly-assigned U.S. Pat. Nos. 9,295,524, 9,339,344, 9,358,074, and 9,452,019, the complete disclosures of which are hereby incorporated by reference in their entirety for all purposes.

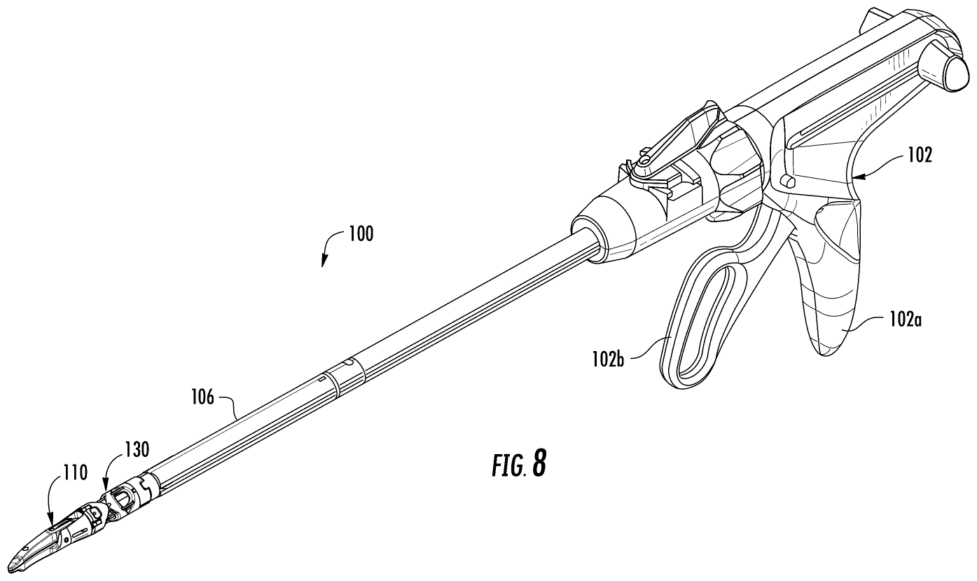

FIG. 8 is a perspective view of another illustrative surgical instrument 100 that may incorporate the end effectors described above in accordance with certain embodiments of the present disclosure. As shown, surgical instrument 100 includes a handle assembly 102, and an end effector 110 mounted on an elongated shaft 106 of the surgical stapling instrument 100 via a clevis 130. End effector 110 includes a first jaw 111 and a second jaw 112. Handle assembly 102 includes a stationary handle 102a and a moveable handle 102b, which serves as an actuator for surgical instrument 100.

In certain embodiments, handle assembly 102 may include input couplers (not shown) instead of, or in addition to, the stationary and movable handles. The input couplers provide a mechanical coupling between the drive tendons or cables of the instrument and motorized axes of the mechanical interface of a drive system. The input couplers may interface with, and be driven by, corresponding output couplers (not shown) of a telesurgical surgery system, such as the system disclosed in U.S. Pub. No. 2014/0183244A1, the entire disclosure of which is incorporated by reference herein for all purposes. The input couplers are drivingly coupled with one or more input members (not shown) that are disposed within the instrument shaft 106 and end effector 110. Suitable input couplers can be adapted to mate with various types of motor packs (not shown), such as the stapler-specific motor packs disclosed in U.S. Pat. No. 8,912,746, or the universal motor packs disclosed in U.S. Pat. No. 8,529,582, the disclosures of both of which are incorporated by reference herein in their entirety for all purposes. Further details of known input couplers and surgical systems are described, for example, in U.S. Pat. No. 8,597,280, U.S. Pat. No. 7,048,745, and U.S. Pat. No. 10,016,244. Each of these patents is hereby incorporated by reference in its entirety for all purposes.

Actuation mechanisms of surgical instrument 100 may employ drive cables that are used in conjunction with a system of motors and pulleys. Powered surgical systems, including robotic surgical systems that utilize drive cables connected to a system of motors and pulleys for various functions including opening and closing of jaws, as well as for movement and actuation of end effectors are well known. Further details of known drive cable surgical systems are described, for example, in U.S. Pat. No. 7,666,191 and U.S. Pat. No. 9,050,119 both of which are hereby incorporated by reference in their entireties for all purposes. While described herein with respect to an instrument configured for use with a robotic surgical system, it should be understood that the wrist assemblies described herein may be incorporated into manually actuated instruments, electro-mechanical powered instruments, or instruments actuated in any other way.

Hereby, all issued patents, published patent applications, and non-patent publications that are mentioned in this specification are herein incorporated by reference in their entirety for all purposes, to the same extent as if each individual issued patent, published patent application, or non-patent publication were specifically and individually indicated to be incorporated by reference.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present disclosure is intended to embrace all such alternatives, modifications and variances. As well, one skilled in the art will appreciate further features and advantages of the present disclosure based on the above-described embodiments. Accordingly, the present disclosure is not to be limited by what has been particularly shown and described, except as indicated by the appended claims.

What is claimed is:

1. A surgical instrument for performing a surgical function comprising:
an elongate shaft having a longitudinal axis;
an end effector mounted on the elongate shaft and including first and second opposable jaws, wherein at least the a first jaw is movable relative to the shaft, wherein the first jaw comprises a non-linear slot and the second jaw comprises a linear slot, wherein the non-linear slot is a compound slot that comprises a proximal portion and a distal portion, wherein the distal portion comprises a proximal end adjacent the proximal portion, wherein the distal portion is continuously curved from the proximal end to a distal end of the compound slot and the proximal portion is linear;
a pin positioned within the non-linear slot and the linear slot such that translation of the pin through the non-linear slot and the linear slot rotates the first jaw between open and closed positions; and
wherein the first and second jaws define a first angle therebetween in the open position, and wherein the first and second jaws define a second angle therebetween when the pin is located at a junction between the distal and proximal portions of the compound slot, wherein the second angle is about 20% or less of the first angle, such that after the pin travels past the junction a higher mechanical advantage is provided to ensure that sufficient clamping force is achieved before the surgical function is performed.

2. The surgical instrument of claim 1, wherein the distal portion of the compound slot is shaped such that the first and second jaws apply a constant grip force therebetween as the pin is translated proximally through said distal portion and the proximal portion of the compound slot is shaped to provide a non-constant grip force between the first and second jaws as the pin is translated through said proximal portion.

3. The surgical instrument of claim 1, wherein the distal portion of the compound slot is shaped such that a force applied by movement of the first jaw is proportional to a force applied to the pin as the pin is translated distally through said distal portion.

4. The surgical instrument of claim 1, wherein the proximal portion of the compound slot is shaped such that a force applied by movement of the first jaw increases non-proportionally relative to the force applied to the pin as the pin is translated proximally through said distal portion.

5. The surgical instrument of claim 1, wherein the second jaw is a stationary jaw supported on a clevis coupled to the elongate shaft and the first jaw is a movable jaw and wherein the end effector further comprises a pivot pin, wherein translation of the pin within the linear slot rotates the movable jaw about the pivot pin.

6. The surgical instrument of claim 1, wherein the non-linear slot has a shape with the formula:
$$R ( θ ) = \frac{( a - b )}{λ} θ + b$$
wherein R is a profile of the non-linear slot as a function of an angle θ between the first and second jaws, a is a distance between the pivot pin and the pin when the first and second jaws are in the open position, b is a distance between the pivot pin and the pin when the first and second jaws are in the closed position, λ an angle between the first and second jaws in the open position, and θ has a range from 0 to λ.

7. The surgical instrument of claim 1, wherein at least one of the first and second jaws includes an electrode.

8. The surgical instrument of claim 1, further comprising an actuation mechanism configured to translate the pin through the linear and non-linear slots, wherein the actuation mechanism is coupled to a control device of a robotic surgical system.

9. The surgical instrument of claim 8, wherein the robotic surgical system comprises a manipulator assembly having an arm that supports the instrument.

10. The surgical instrument of claim 9, wherein the robotic surgical system further comprises a surgeon console having an input device to control the instrument.

11. A surgical instrument comprising:
an elongate shaft having a longitudinal axis;
an end effector mounted on the elongate shaft and including first and second opposable jaws, wherein at least a first jaw is movable relative to the shaft, wherein the first jaw comprises a non-linear slot and the second jaw comprises a linear slot,
a pin positioned within the non-linear slot and the linear slot such that translation of the pin through the non-linear slot and the linear slot rotates the first jaw between open and closed positions; and
wherein the non-linear slot has a shape with the formula:
$$R ( θ ) = \frac{( a - b )}{λ} θ + b$$
wherein R is a profile of the non-linear slot as a function of an angle θ between the first and second jaws, a is a distance between the pivot pin and the pin when the first and second jaws are in the open position, b is a distance between the pivot pin and the pin when the first and second jaws are in the closed position, λ an angle between the first and second jaws in the open position, and θ has a range from 0 to λ.

12. The surgical instrument of claim 11, wherein the non-linear slot is a compound slot that comprises a proximal portion and a distal portion, wherein the distal portion comprises a proximal end adjacent the proximal portion, wherein the distal portion is continuously curved from the proximal end to a distal end of the compound slot and wherein the distal portion of the compound slot is shaped such that the first and second jaws apply a constant grip force therebetween as the pin is translated proximally through said distal portion, and the proximal portion is linear.

13. The surgical instrument of claim 11, further comprising an actuator coupled to the pin and configured to translate the pin through the first and second slots.

14. The surgical instrument of claim 13, wherein the second jaw is a stationary jaw supported on a clevis and the first jaw is a movable jaw and wherein the end effector further comprises a pivot pin, wherein translation of the pin within the second slot rotates the movable jaw about the pivot pin.

15. The surgical instrument of claim 11, wherein a junction is located at a proximal end of the distal portion of the compound slot and at a distal end of the proximal portion of the compound slot.

16. The surgical instrument of claim 11, wherein the first and second jaws apply a constant grip force therebetween when the pin is being translated while distal to the junction and the first and second jaws apply a non-constant grip force therebetween when the pin is being translated while proximal of the junction.

17. The surgical instrument of claim 11, wherein the first and second jaws apply a stronger grip force when the pin is being translated while proximal to the junction than when the pin is being translated while distal to the junction.

18. The surgical instrument of claim 11, wherein the robotic surgical system comprises a manipulator assembly having an arm that supports the instrument.

19. The surgical instrument of claim 18, wherein the robotic surgical system further comprises a surgeon console having an input device to control the instrument.

20. A robotic surgical system comprising:
a manipulator assembly having an arm that supports a surgical instrument for performing a surgical function; and
a surgeon console having an input device to control the movement of the surgical instrument via the arm;
the surgical instrument comprising:
an elongate shaft having a longitudinal axis;
an end effector mounted on the elongate shaft and including first and second opposable jaws, wherein at least the a first jaw is movable relative to the shaft, wherein the first jaw comprises a non-linear slot and the second jaw comprises a linear slot, wherein the non-linear slot is a compound slot that comprises a proximal portion and a distal portion, wherein the distal portion comprises a proximal end adjacent the proximal portion, wherein the distal portion is continuously curved from the proximal end to a distal end of the compound slot and the proximal portion is linear;
a pin positioned within the non-linear slot and the linear slot such that translation of the pin through the non-linear slot and the linear slot rotates the first jaw between open and closed positions; and
wherein the first and second jaws define a first angle therebetween in the open position, and wherein the first and second jaws define a second angle therebetween when the pin is located at a junction between the distal and proximal portions of the compound slot, wherein the second angle is about 20% or less of the first angle, such that after the pin travels past the junction a higher mechanical advantage is provided to ensure that sufficient clamping force is achieved before the surgical function is performed.
